(19) United States
(12) Patent Application Publication
(10) Pub. No.: US 2018/0016202 A1
(43) Pub. Date: Jan. 18, 2018

(54) OBSCURANT COMPOSITIONS

(71) Applicant: JOHN L. LOMBARDI, Tucson, AZ (US)

(72) Inventor: JOHN L. LOMBARDI, Tucson, AZ (US)

(21) Appl. No.: 15/472,126

(22) Filed: Mar. 28, 2017

Related U.S. Application Data
(63) Continuation-in-part of application No. 14/800,534, filed on Jul. 15, 2015.
(60) Provisional application No. 62/024,875, filed on Jul. 15, 2014.
(60) Provisional application No. 62/473,926, filed on Mar. 20, 2017.

(51) Int. Cl. C06D 3/00; C07D 307/33; C07C 233/25; C08K 13/02; C06B 29/08

(52) U.S. Cl. CPC C06D 3/00; C08K 13/02; C06B 29/08; C07C 233/25; C07D 307/33

(57) ABSTRACT

An enhanced pyrotechnic composition including an obscurant, a fuel, an oxidizer, and a nonivamide-cyclic anhydride adduct.

### CROSS REFERENCE TO RELATED APPLICATIONS

This Application is a Continuation-In-Part of U.S. Non-Provisional Patent Application having Ser. No. 14/800,534, filed on Jul. 15, 2015, which claimed priority from U.S. Provisional Patent Application having Ser. No. 62/024,875, filed on Jul. 15, 2014, which are hereby incorporated by reference herein. This Application further claims priority from a U.S. Provisional Patent Application having Ser. No. 62/473,926, filed on Mar. 20, 2017, which is also hereby incorporated by reference herein.

### FIELD OF THE DISCLOSURE

Compositions of matter are disclosed, wherein those compositions of matter comprise an oleo resin in combination with an obscurant formulation.

### BACKGROUND OF THE DISCLOSURE

Naturally occurring obscurants, such as fog, snow, or rain are unpredictable, and in many geographic locations, infrequent. As such, artificial obscurants are common in military operations. Artificial obscurants may be selected to block electromagnetic radiation in the visible spectrum (approximately 0.38 μm to approximately 0.78 μm), the near infrared spectrum (NIR) (approximately 0.78 μm to approximately 3 μm), the mid infrared spectrum (MIR) (approximately 3 μm to approximately 50 μm), the far infrared spectrum (FIR) (approximately 50 μm to approximately 1000 μm), or a combination thereof.

Modified versions of traditional weapon delivery systems are used to deploy obscurants in the field. The explosive payload of various munitions, including grenades, rockets, and other artillery, are removed and replaced with a payload comprising an obscurant composition. The use of a particular munition type depends on the particular use. For example, obscurant grenades may be employed in small scale tactical combat operations. Rockets, mortars, or large scale artillery carrying obscurant composition payloads may be used to conceal or protect large areas, such as air fields or large scale troop movements. Upon ignition or detonation, the obscurant composition burns to produce a cloud of smoke that blocks a given spectrum of light.

Obscurants are compounds that are capable of marking, blocking, scattering, and/or absorbing light and are often leveraged in military operations. Obscurants can aid with friendly operations by, for example, providing cover for troop movement, concealing the location and size of friendly forces, concealing valuable facilities from enemy forces, and marking targets. Obscurants can also obstruct and disrupt enemy operations by, for example, interfering with enemy communications and coordination.

### BRIEF DESCRIPTION OF THE DRAWINGS

The disclosure will be better understood from a reading of the following detailed description taken in conjunction with the drawings in which like reference designators are used to designate like elements, and in which:

FIG. 1 is a FTIR spectra of Nonivamide Reactant;

FIG. 2A is a ¹NMR of Nonivamide Reactant;

FIG. 2B is a ¹³C NMR of Nonivamide Reactant;

FIG. 3A is a ¹H NMR of Nonivamide-Maleic Anhydride Adduct;

FIG. 3B is a ¹³C NMR of Nonivamide-Maleic Anhydride Adduct;

FIG. 4A is a ¹H NMR of Nonivamide-Phthalic Anhydride Adduct; and

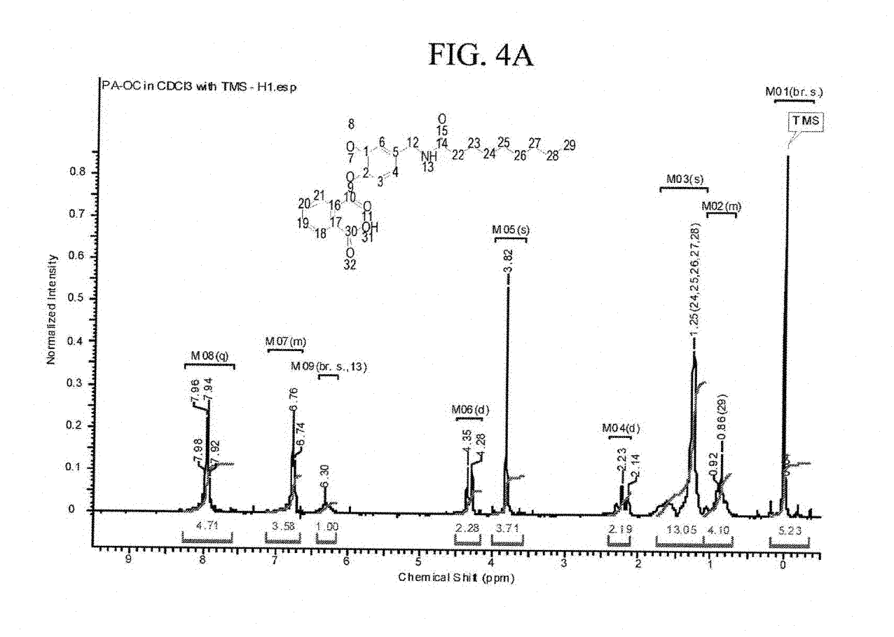

FIG. 4B is a ¹³C NMR of Nonivamide-Phthalic Anhydride Adduct;

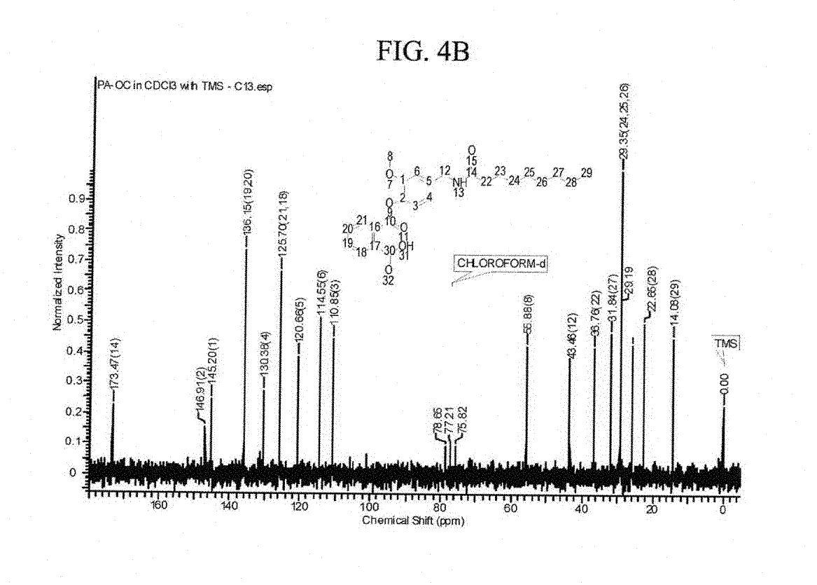

FIG. 5 illustrates Relative Apparent Extinction Coefficient of unmodified MATEAB control and various candidate imide modified smoke formulations as a function of incident light wavelength obtained during pellet combustion trials within a smoke box containing a NIR—Visible Spectrometer sensor. (All samples tested at 20° C. and 20 percent relative humidity.);

FIG. 6 Illustrates Relative Apparent Extinction Coefficient of unmodified MATEAB control, candidate phthalimide candidate Guanidine Carbonate modified smoke formulations as a function of incident light wavelength obtained during pellet combustion trials within a smoke box containing a NIR—Visible Spectrometer sensor. (All samples tested at 20° C. and 20% relative humidity.)

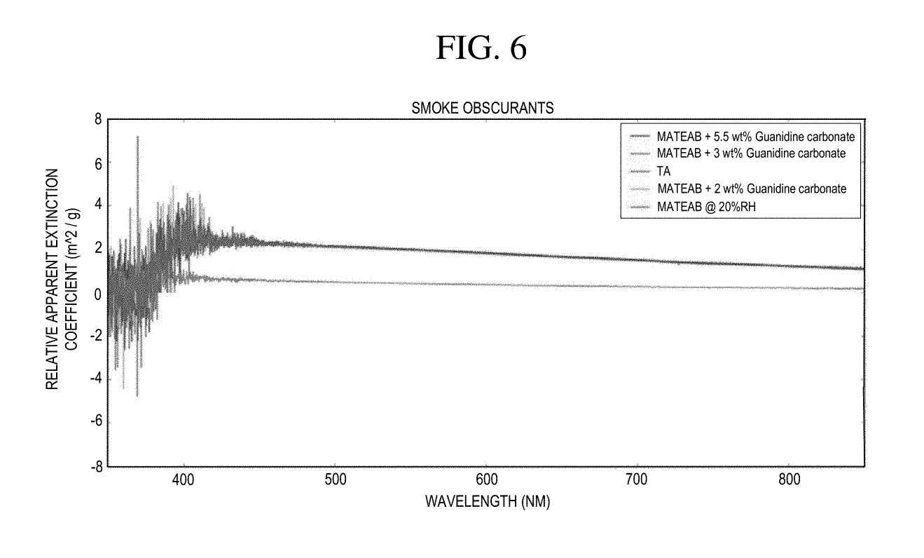

FIG. 7 illustrates Relative Apparent Extinction Coefficient of unmodified MATEAB control as well as various candidate Guanidine Carbonate & Phthalimide modified smoke formulations as a function of incident light wavelength obtained during pellet combustion trials within a smoke box containing a NIR—Visible Spectrometer sensor. (Samples tested at 20 & 80% Relative Humidity (RH) and 20° C.); and

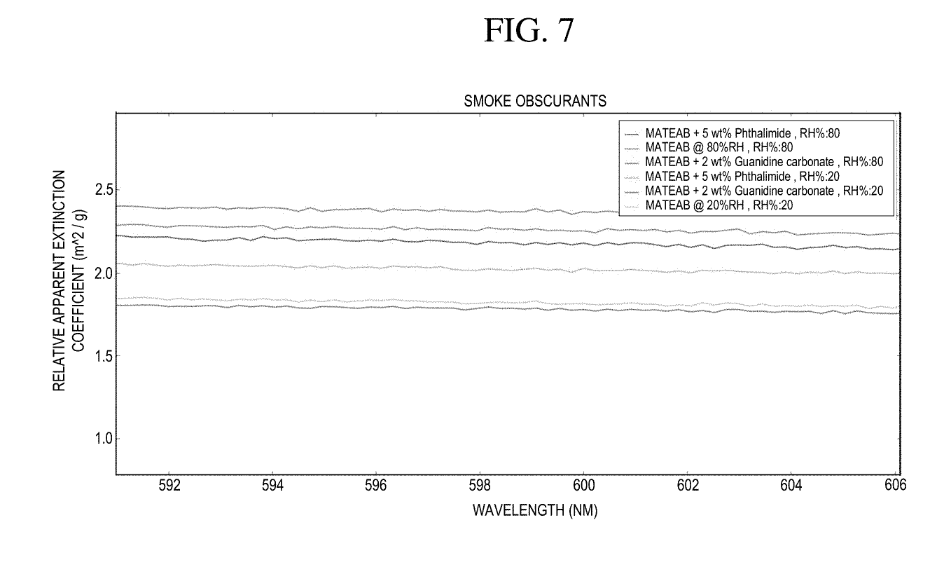

FIG. 8 illustrates a Comparison of unmodified MATEAB control as well as Guanidine Phosphate (monobasic & dibasic) and PEPA phosphate modified MATEAB smoke obscuration versus unmodified MATEAB control. (Samples tested at 20° C. and 20% relative humidity).

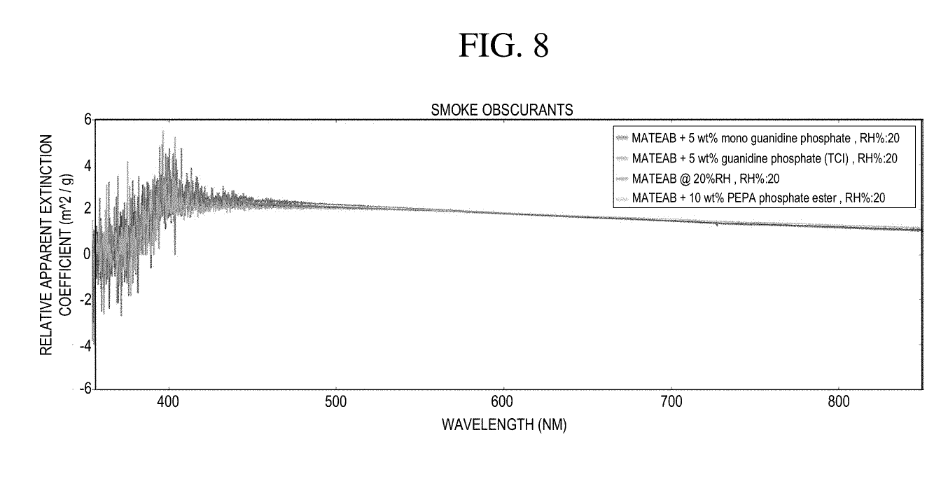

### DETAILED DESCRIPTION OF THE PREFERRED EMBODIMENTS

Applicant's disclosure is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present disclosure. Thus, appearances of the phrases “in one embodiment,” “in an embodiment,” and similar language throughout this specification may, but do not necessarily, all refer to the same embodiment.

The described features, structures, or characteristics of the disclosure may be combined in any suitable manner in one or more embodiments. In the following description, numerous specific details are recited to provide a thorough understanding of embodiments of the disclosure. One skilled in the relevant art will recognize, however, that the disclosure may be practiced without one or more of the specific details, or with other methods, components, materials, and so forth. In other instances, well-known structures, materials, or operations are not shown or described in detail to avoid obscuring aspects of the disclosure.

Obscurants are compounds that are capable of marking, blocking, scattering, and/or absorbing light and are often leveraged in military operations. Obscurants can aid with friendly operations by, for example, providing cover for troop movement, concealing the location and size of friendly forces, concealing valuable facilities from enemy forces, and marking targets. Obscurants can also obstruct and disrupt enemy operations by, for example, interfering with enemy communications and coordination.

Nonivamide and related oleoresin capsaicin (OC) compounds are known non-lethal agents employed for protective as well as riot and crowd control purposes. Such agents have found increasing use given the recent concerns regarding the toxicity of o-chlorobenzylidene malononitrile (CS) and related conventional synthetic tear gas compositions. Nonivamide and OC are typically dissolved within solvents (e.g. alcohols, glycols, acetone, methylene chloride, ketones etc.) and contacted upon assailants via spraying or projectile means. They are typically not dispersed and vaporized via pyrotechnic propellants given their inherent limited thermal stability and susceptibility towards decomposition when heated at high temperatures associated with pyrotechnic propellant combustion (e.g. sugar-chlorate, nitrate or perchlorate pyrotechnic systems found within grenades, smoke pots etc.). In comparison to the former, pyrotechnic devices offer a better means of delivering OC agent in a controlled and more prolonged fashion. Hence there is considerable advantages towards producing versions of OC which are more thermally stable and hence more compatible with conventional pyrotechnic propellants.

One means of stabilizing OC entails converting the phenolic hydroxyl on the molecule to a borate, phosphate or carboxylate ester. Such esterification methods are well known to those skilled in the art and include direct esterification between the OC phenolic hydroxyl and boric, phosphoric or carboxylic acid accompanied by water condensation by-product removal, reaction of phenolic with corresponding acid halide, acid anhydride or transesterification with borate, phosphate or carboxylic derived ester having favorable leaving groups. Such reactions may be conducted neat or within a solvent. Examples of ester protected OC reactions are provided below.

### Example 1

OC Maleate Ester Synthesis

About 0.6230 g of N-vanillylnonivamide (Nonivamide, synthetic capsaicin) (TCI America) was reacted neat at 80° C. with a slight molar excess (0.2097 g) of Maleic Anhydride (Huntsman Chemical) for 15 minutes followed by cooling to room temperature producing a transparent yellow colored viscous oil product. The reaction progress was monitored using ¹H and ¹³C Nuclear Magnetic Resonance (NMR) Spectroscopy whereby downfield shifts in the phenolic aromatic ring hydroxyl group proton and associated carbon atom peaks was observed after maleic anhydride addition to the melt. (See spectra and Table 4 below for NMR Spectral Shifts obtained within deuterochloroform solution using an Anasazi Instruments 90 MHz NMR Spectrometer.)

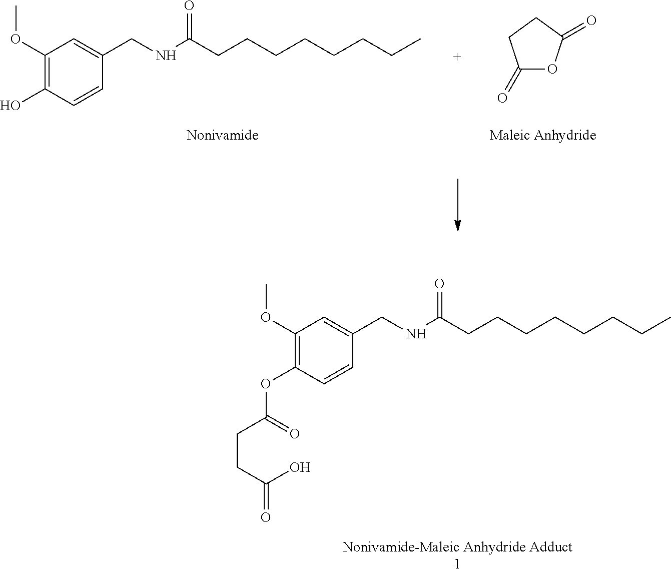 [SMILES: CCCCCCCCC(=O)NCc1ccc(O)c(OC)c1 ; O=C1CCC(=O)O1 ; CCCCCCCCC(=O)NCc1ccc(OC(=O)CCC(=O)O)c(OC)c1]

### Example 2

OC Phthalate Ester Synthesis

A similar procedure was employed as Example 1 except 0.3580 g of N-vanillylnonivamide (TCI America) was reacted neat at 90° C. with a slight molar excess (0.185 g) of Phthalic Anhydride (Stepan Chemical Company) for 10 minutes which ultimately yielded an off white solid product upon cooling to ambient temperature. The reaction progress was monitored using 1H and 13C Nuclear Magnetic Resonance (NMR) Spectroscopy whereby downfield shifts in the phenolic aromatic ring hydroxyl group proton and associated carbon atom peaks was observed after phthalic anhydride addition to the melt. (See spectra and Table 4 below for NMR Spectral Shifts obtained within deuterochloroform solution using an Anasazi Instruments 90 MHz NMR Spectrometer.)

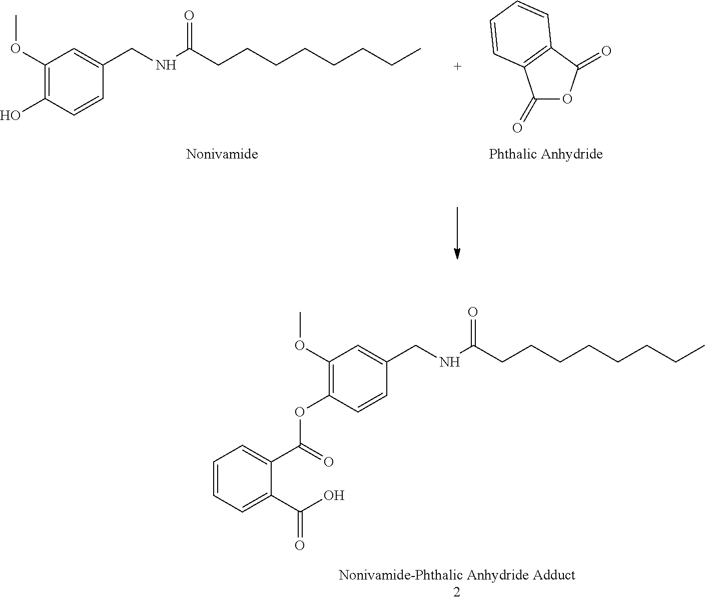 [SMILES: CCCCCCCCC(=O)NCc1ccc(O)c(OC)c1 ; O=C1OC(=O)c2ccccc21 ; CCCCCCCCC(=O)NCc1ccc(OC(=O)c2ccccc2C(=O)O)c(OC)c1]

### Example 3

### OC Succinate Ester Synthesis

The same neat melt reaction approach was employed as the former except 0.3118 g of N-vanillylnonivamide (TCI America) was reacted with 0.1099 g of Succinic Anhydride (TCI America) at 110° C. for 15 minutes. A white colored solid product resulted upon cooling the melt to room temperature and NMR Spectral analysis verified phenolic hydroxyl reaction with this acid anhydride.

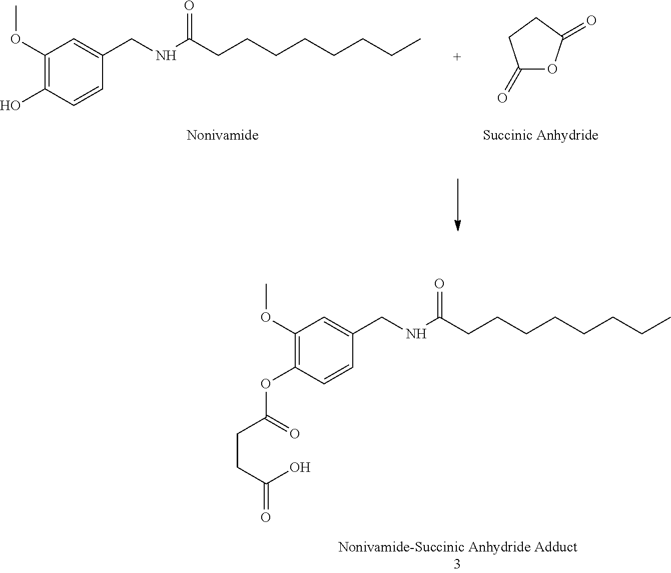 [SMILES: CCCCCCCCC(=O)NCc1ccc(O)c(OC)c1 ; O=C1CCC(=O)O1 ; CCCCCCCCC(=O)NCc1ccc(OC(=O)CCC(=O)O)c(OC)c1]

TABLE 4

| ¹H Peak Chemical | ¹³C Peak Chemical |
|---|---|
| Shift (ppm) | Shift (ppm) |
| Nonivamide Reactant 6.14 | 145.36 |
| Nonivamide - Maleic 6.36 | 147.05, 145.30 |
| Anhydride Adduct | |
| Nonivamide - Phthalic 6.30 | 146.91, 145.20 |
| Anhydride Adduct | |

As those having skill in the art will appreciate, Applicant's Nonivamide/Maleic Anhydride Adduct 1 reacts with ambient water in the atmosphere, i.e. ambient humidity, fog, mist, precipitation, and the like, to cleave the ester adduct to regenerate in the ambient air the original nonivamide non-lethal, crowd control, agent.

As those having skill in the art will appreciate, Applicant's Nonivamide/Phthalic Anhydride Adduct 2 reacts with ambient water in the atmosphere, i.e. ambient humidity, fog, mist, precipitation, and the like, to cleave the ester adduct to regenerate in the ambient air the original nonivamide non-lethal, crowd control, agent.

As those having skill in the art will appreciate, Applicant's Nonivamide/Succinic Anhydride Adduct 3 reacts with ambient water in the atmosphere, i.e. ambient humidity, fog, mist, precipitation, and the like, to cleave the ester adduct to regenerate in the ambient air the original nonivamide non-lethal, crowd control, agent.

Obscurant compositions currently used by the military include white phosphorous (WP), red phosphorous (RP), hexachloroethane (HC), and terephthalic acid (TA). These obscurants exhibit a number of undesirable properties, including high toxicity, poor shelf life, and high burn temperatures.

When white phosphorous burns in air, it produces a hydroscopic compound, diphosphorus pentoxide. As the diphosphorus pentoxide absorbs moisture from the atmosphere, small airborne droplets of phosphoric acid are formed. White phosphorous, however, is pyrophoric at relatively low temperatures. It will ignite in air at about 30° C., making it hazardous to handle, store, and transport.

Red phosphorous (RP) has largely replaced white phosphorous for obscurant purposes. Over time red phosphorous slowly degrades to highly toxic phosphine gas, a pyrophoric gas that can self-ignite when mixed with air.

Elemental phosphorous-based obscurants (both red and white) have a number of other drawbacks.

First, because they burn at high temperatures (>500° C.) and have a high flame front, they pose the risk of burning nearby personnel or noncombatants, damaging nearby buildings or equipment, and igniting secondary fires. Second, the resulting obscurant cloud is composed of acidic water vapor, which is a respiratory irritant. Inhalation of this vapor can pose a health threat to nearby personnel and civilians.

Hexachloroethane-based obscurant compositions (HC) are produced by combining hexachloroethane, aluminum powder, and zinc oxide. Upon combustion, the mixture produces zinc chloride, which in turn absorbs moisture from the air to form an obscurant cloud. The zinc chloride in the resulting cloud is lethal if inhaled, capable of causing gross pathological pulmonary injuries and death due to pulmonary edema. Hexachloroethane-based obscurants, like the phosphorous-based variations, also have a high combustion temperature.

Terephthalic acid-based obscurant formulations (TA & TA/PE), unlike elemental phosphorous-based and hexachloroethane-based obscurants, produce a nontoxic smoke. (See Table below for conventional TA/PE smoke formulation.) However, terephthalic acid-based obscurants have limited obscuring properties as compared to WP, RP, or HC. Furthermore, given its high inherent combustion temperature (e.g. typical peak exotherm T>425° C.), slag forming alkali and alkaline earth carbonate and bicarbonate coolants are often incorporated into conventional TA/PE formulations to lower its pyrolysis temperature and prevent unwanted fires.

Besides forming undesirable slag ash, these coolants also lower the mass fraction of obscurant within a formulation and thereby limits the overall yield from a given smoke producing device. For example as can be seen upon comparison between Tables I & IV below, conventional TA/PE smoke formulation is limited to containing only 36.99 weight percent TA obscurant given its need for 7.01 weight percent magnesium carbonate and sodium bicarbonate coolant additive whereas the novel melamine based MATEAB formulation does not require such coolants and is therefore able to contain 53.01 weight percent melamine, acetoguanamine and triethanolamine borate obscurant phase. Similar high obscurant concentrations can be seen in smoke compositions listed in Examples I through V below of the present disclosure having weight percent obscurants totaling 54.07, 55.19, 57.32, 54.32 & 56.05, respectively, given that these too exhibit inherently lower peak combustion temperatures compared to the conventional TA formulation.

The rate or production of obscuring smoke produced by conventional obscurants is largely dependent on the packing density of the components. Obscurant devices with higher packing densities produce obscurant smoke at a higher rate. Packing densities, however, are difficult to control in practice and generally result in inconsistent results. Moreover, obscurant devices with varying rates of smoke production (i.e., an initial high production rate followed by a slower sustaining rate) are likewise difficult to produce with any reliability by varying packing densities.

Accordingly, it would be an advance in the state of the art to provide an obscurant composition for use in traditional applications that (i) burns at a lower temperature than existing compositions, (ii) produces a non-toxic obscurant cloud, (iii) equals or outperforms existing compositions in obscuring performance, (iv) produces smoke in higher yield, (v) remains stable during long term storage, (vi) can have its burn rate adjusted via controlling the relative concentration of obscurant components (e.g. phthalimide and/or phthalimide/acetoguanamine content) within the formulation (vii) is capable of producing variable smoke production rates without relying on packing density, (viii) is produced from nontoxic components, (ix) is environmentally friendly, and (x) is cost competitive with existing obscurants.

This disclosure is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present disclosure. Thus, appearances of the phrases “in one embodiment,” “in an embodiment,” and similar language throughout this specification may, but do not necessarily, all refer to the same embodiment.

The described features, structures, or characteristics of the disclosure may be combined in any suitable manner in one or more embodiments. In the following description, numerous specific details are recited to provide a thorough understanding of embodiments of the disclosure. One skilled in the relevant art will recognize, however, that the disclosure may be practiced without one or more of the specific details, or with other methods, components, materials, and so forth. In other instances, well-known structures, materials, or operations are not shown or described in detail to avoid obscuring aspects of the disclosure.

Applicant has developed an obscurant formulation based on nontoxic components that is capable of producing a nontoxic cloud at low combustion temperatures (T<400° C.) with excellent obscuring properties and higher yield than conventional TA formulations without the need for slag forming coolant addition to its formulation. In one embodiment, Applicant's formulation comprises a powdered melamine/obscurant additive combined with a sucrose/chlorate fuel-oxidizer system. Upon ignition, the heat produced by the combustion of the fuel causes the triazine, imide, TEAB blend to sublime, producing an obscurant smoke. Such a blend sublimes at a relatively low temperature and re-condenses as smoke aerosol particles, therefore the burn temperature of Applicant's formulation is lower than conventional obscurants and produces a minimal flame front.

By varying the phthalimide or guanidine carbonate content, it is possible to produce smoke formulations having a wide variety of burn rates. From the results in Table VI below, it can be seen that a 4.76 weight percent phthalimide addition to MATEAB smoke (Example II) increases its burn time from 16 (Table IV unmodified MATEAB formulation) to 26.2 seconds (4.72 wt. % Phthalimide modified MATEAB) whereas complete replacement of acetoguanamine with phthalimide (Example I formulation) produced a 16.8 second burn time. A 4.72 weight % phthalimide addition (Example II) or 2.92 weight % Guanidine Carbonate (Example IV) addition were also separately found to enhance the obscuring properties (as measured in terms of relative apparent extinction coefficients) of MATEAB based smoke formulations.

An embodiment of the present disclosure provides a composition of matter capable of producing a nontoxic smoke upon combustion. The composition of matter comprises a smoke formulation. In certain embodiments, Applicant's smoke formulation comprises triazines (i.e. melamine, acetoguanamine, benzoguanamine or blends thereof) and optionally an imide (i.e. glutarimide, succinimide and/or its alkyl and alkenyl substituted derivatives, tetrachlorophthalimide, tetrabromophthalimide, phthlimide and/or its derivatives, trimellitimide and/or its esters, amides or salts, alkanolamine borate (i.e. triethanolamine borate (TEAB), triisopropanolamine borate), cyclic phosphate ester (i.e. pentaerythritol phosphate alcohol, propylene glycol phosphate, neopentyl glycol phosphate and/or its blends thereof), cationic amine salt (i.e. tetraalkyl ammonium or mixed tetraalkyl/aryl ammonium, imidazolium or guanidinium nitrate, acetate, benzoate, carbonate, phosphate, polyphosphate, borophosphate, oxalate & sulfamate salts), cyclic ester (i.e. lactide, glycolide, caprolactone, gluconolactone, butyrolactone), organic carbonate (i.e. trimethylene carbonate, ethylene carbonate, propylene carbonate, glycerin carbonate and its ester derivatives) or blends thereof.

The esters of glycerin carbonate include the following compounds and methods to prepare same.

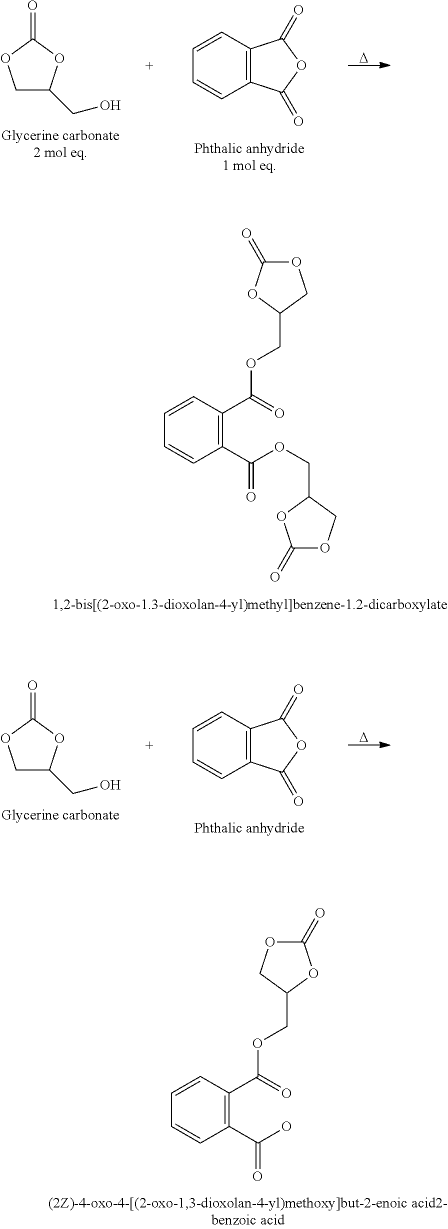

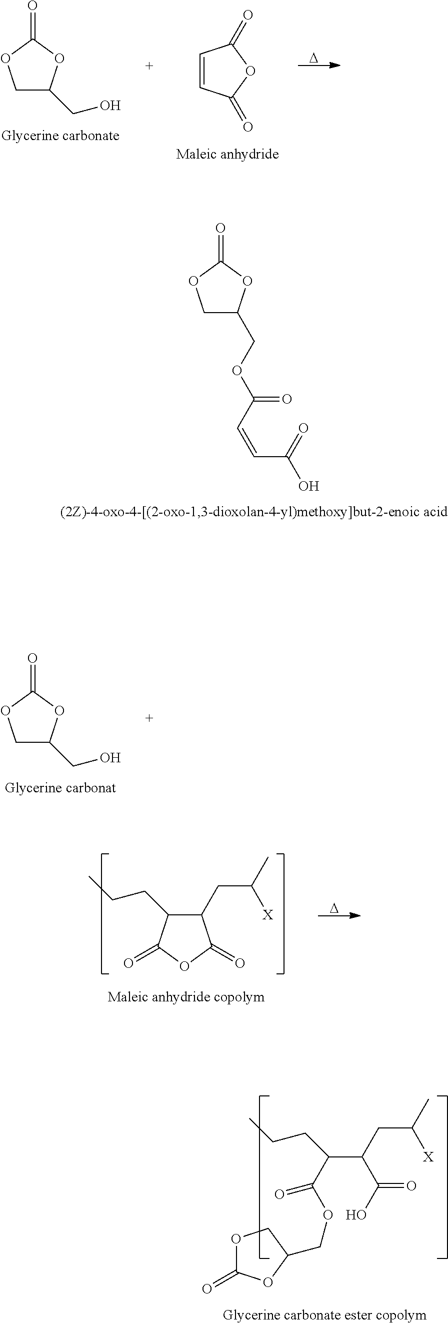

As can be seen from the obscurant properties in the tables below, melamine is of particular interest given its higher refractive index relative to TA (e.g. Melamine n_{D}=1.872 versus TA n_{D}=1.648 respectively) and hence exhibits a higher capacity to interact with incident light. The below tables also indicate that melamine and phthalimide have lower enthalpies of vaporization and sublimation requiring less heat for its efficient sublimation and re-condensation into aerosol smoke particles compared to TA. Finally, melamine and phthalimide exhibit low acute toxicity and are relatively inexpensive commodity chemicals having low acute toxicity.

The differences in obscuration properties between conventional TA versus melamine based MATEAB smoke formulations can readily be seen in the spectral results below measured by Professor Charles Bruce from the Department of Physics at New Mexico State University whereby TA and MATEAB melamine obscurant based smokes were found to exhibit mass-normalized extinction efficiencies measured at a wavelength of 6.3 μm of 3.57±2.9 m2/g versus 5.1-5.46 m2/g respectively. Given that extinction efficiency is a base ten exponent, this indicated that the melamine MATEAB based smokes had in excess of a 33 fold higher obscuration performance compared to conventional TA smoke at this wavelength.

The composition of matter further comprises an oxidizer selected from the group consisting of potassium chlorate and sodium chlorate. Alternative pyrotechnic oxidizers including but not limited to guanidine nitrate, alkali/alkaline earth nitrates, alkali/alkaline earth perchlorates, may be used alone or blended with the aforementioned chlorates. The smoke formulation further comprises a fuel. The fuel comprises sulfur, charcoal, silicon, silicone, trioxane, hexamine, metaldehyde, glycols, polyols, cellulose, styrenic polymers, maleic anhydride copolymers, epoxy resin, nitrocellulose, starch, nitrostarch, pentaerythritol, sorbitol, xylitol, glucose, fructose, sucralose, dextrose, lactose, sucrose or similar carbohydrate and blends thereof. The smoke formulation may also comprise filler including silica, alumina, clay, microcrystalline cellulose, diatomaceous earth or similar particulate which can serve as an adsorbent or adsorbent support for nonivamide, OC and its ester derivatives. Unmodified nonivamide or OC incapacitant and/or its esterified derivatives.

Preferred compositions comprise blends between (10-60 weight %) melamine, (0.000001-40 weight %) phthalimide, (25-35 weight %) potassium chlorate, (10-20 weight %) sucrose and optionally with or without (0-25 weight %) triethanolamine borate and (0-25 weight %) acetoguanamine. The following Examples are presented to further illustrate to persons skilled in the art how to make and use the disclosure. These Examples are not intended as a limitation, however, upon the scope of Applicant's disclosure.

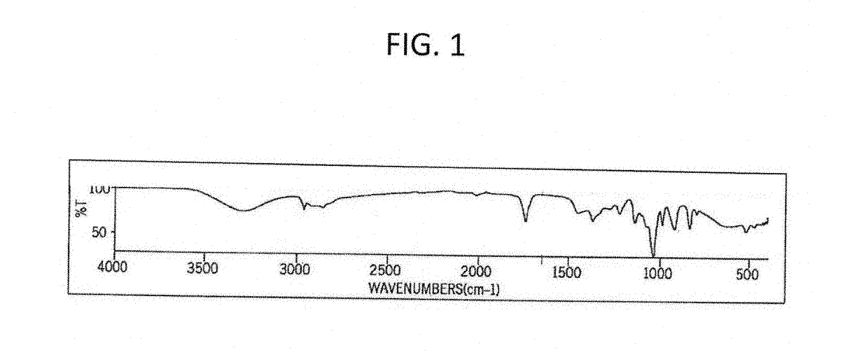

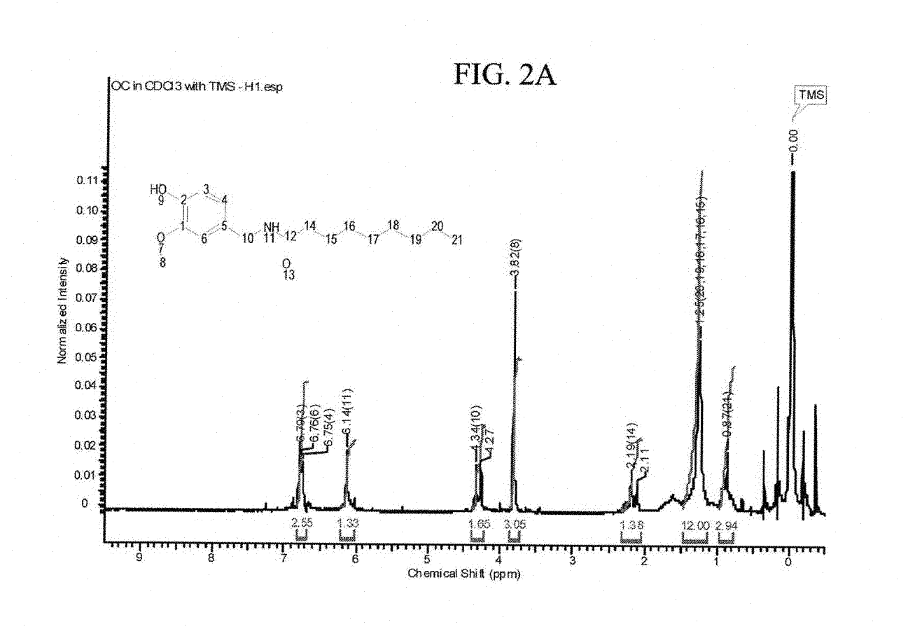

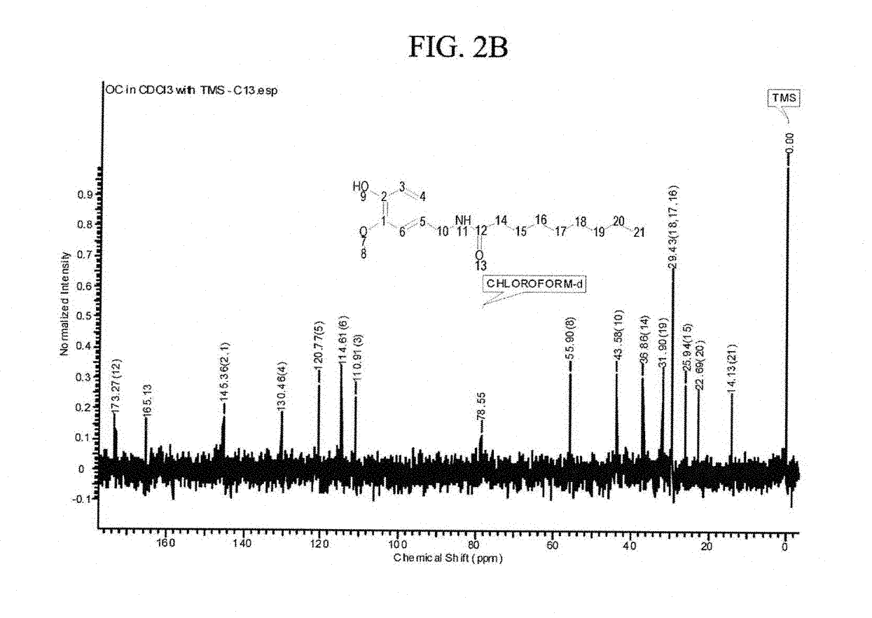

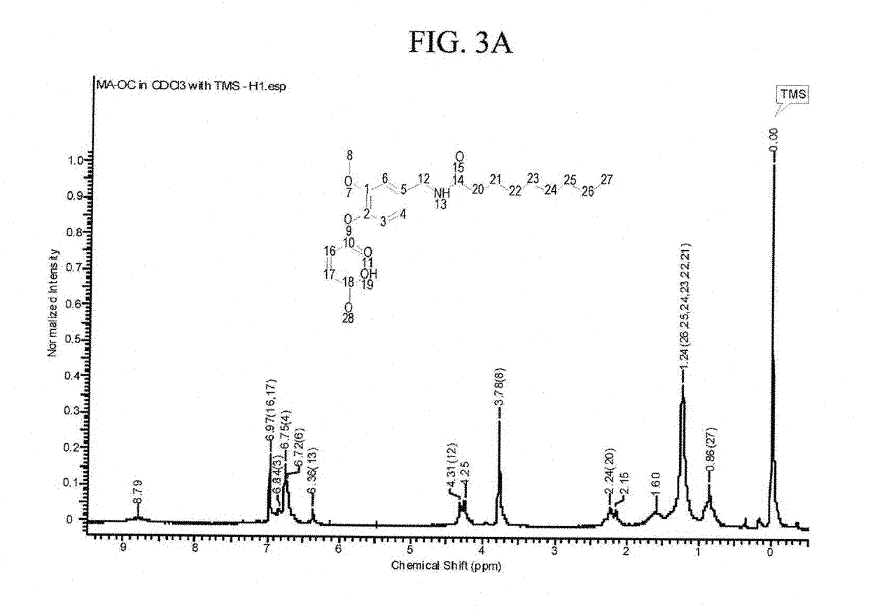

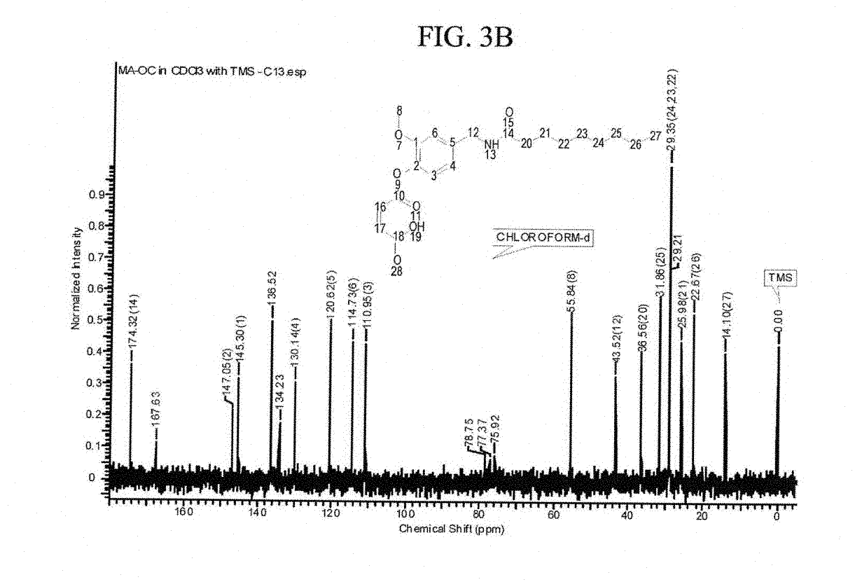

Subject compositions were separately batched within acetone vehicle followed by mixing within a Hobart planetary mixer and later carefully dried within a convection oven heated at 70° C. Pellets were compacted from the dried mixtures using a Carver Hydraulic Press operating at 20,000 lb load for 20 second duration. Pellets were separately combusted within a smoke box and apparent relative extinction coefficients were calculated from NIR Spectral transmittance measurements made using a Thorlabs CCS200 Compact Fiber Visible/Near Infrared (NIR) Spectrometer (500-1000 nm±4 nm spectral sensing wavelength range) sensor present within the smoke box. A typical obscuration plot comparing various candidate smoke formulation pellet combustion trials is presented in FIGS. 1 through 3 below. In general, it can readily be seen that all smokes tested exhibited superior overall obscuration properties compared to conventional TA/PE control smoke. The obscuration properties of the smoke were enhanced at elevated humidity.

TABLE II
Typical Properties of Candidate Sublimable Obscurant Compounds

| | Hildebrand | | | Total # of |
|---|---|---|---|---|
| | Solubility | | Enthalpy of | Hydrogen |
| Obscurant | Parameter | Density | Vaporization | Bonding |
| Compound | δ (cal^{1/2}ml^{-1/2}) | (g/mL) | ΔHᵥₐₚ (cal/g) | Sites^{b} |
| Terephthalic | 12.0 | 1.51 | 99 | 6 |
| Acid (TA) | | | | |
| Melamine | 16.0 | 1.66 | 155 | 12 |
| Acetoguanamine | 13.7 | 1.39 | 135 | 9 |
| Triethanolamine | 8.15 | 1.13 | 58.8 | 4 |
| Borate (TEAB) | | | | |
| Pentaerythritol | 10.3 | 1.35 | 78.4 | 6 |
| Phosphate Alcohol | | | | |
| (PEPA) | | | | |

TABLE III
Typical Properties of Candidate Sublimable Obscurant Compounds

| | Melamine | Acetoguanamine | Phthalimide | TA | |
|---|---|---|---|---|---|
| Melting Point | 354 | 274 | 238 | 300 | ° C. |
| Heat Fusion | | | 28.6 | 21.5 | kJ/mol |
| Heat of | 75.31 | 70.06 | 51.81 | 83.19 | kJ/mol |
| Vaporization | | | | | |
| Heat of Sublimation | 123.3 | | 106.3 | 146.6 | kJ/mol |
| Molecular Weight | 126.12 | 125.132 | 147.13 | 166.133 | g/mol |
| Density | 1.573 | 1.391 | 1.367 | 1.415 | g/cm\hat{ }3 |
| Refractive Index | 1.872 | 1.675 | NA | 1.648 | |

### Example 1

| MPTEAB | |
|---|---|
| Component | Concentration Weight % |
| Potassium Chlorate (Oxidizer) | 32.96 |
| Sucrose (Fuel) | 12.97 |
| Melamine (Obscurant) | 21.36 |
| TEAB (Obscurant) | 16.38 |
| Phthalimide (Obscurant) | 16.33 |

### Example II

| Phthalimide modified MPTEAB | |
|---|---|
| Component | Concentration Weight % |
| Potassium Chlorate (Oxidizer) | 32.16 |
| Sucrose (Fuel) | 12.65 |
| Melamine (Obscurant) | 20.84 |
| TEAB (Obscurant) | 15.98 |
| Acetoguanamine (Obscurant) | 13.61 |
| Phthalimide (Obscurant) | 4.760 |

### Example III

| Phthalimide & PEPA Modified MPTEAB | |
|---|---|
| Component | Concentration Weight % |
| Potassium Chlorate (Oxidizer) | 30.63 |
| Sucrose (Fuel) | 12.05 |
| Melamine (Obscurant) | 19.85 |
| TEAB (Obscurant) | 15.22 |
| Acetoguanamine (Obscurant) | 12.96 |
| Phthalimide (Obscurant) | 4.530 |
| Pentaerythritol Phosphate Alcohol (PEPA) | 4.760 |
| (Obscurant) | |

### Example IV

| Guanidine Carbonate modified MPTEAB | |
|---|---|
| Component | Concentration Weight % |
| Potassium Chlorate (Oxidizer) | 32.78 |
| Sucrose (Fuel) | 12.90 |
| Melamine (Obscurant) | 21.24 |
| TEAB (Obscurant) | 16.29 |
| Acetoguanamine (Obscurant) | 13.87 |
| Guanidine Carbonate (Obscurant) | 2.920 |

### Example V

| MATEAB + Trimellitimide | |
|---|---|
| Component | Concentration Weight % |
| Potassim Chlorate (Oxidizer) | 31.47 |
| Sucrose (Fuel) | 12.48 |
| Melamine (Obscurant) | 20.48 |
| TEAB (Obscurant) | 15.64 |
| Acetogunamine (Obscurant) | 13.41 |
| Trimellitimide (Obscurant)* | 6.52 |

*Trimellitimide was synthesized according to the following procedure:

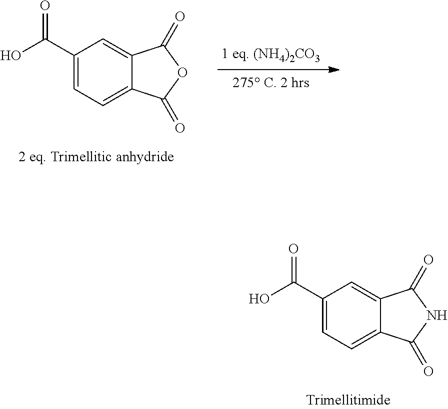 [SMILES: O=C(O)c1ccc2c(c1)C(=O)OC2=O ; O=C(O)c1ccc2c(c1)C(=O)NC2=O]

TABLE VI
Effects of Phthalimide & Guandine Carbonate Additives Upon MATEAB Pyrotechnic
Smoke Formulation Combustion Characteristics

| | | | | Relative |
|---|---|---|---|---|
| | | | Yield | Apparent |
| | Peak | | (Wgt. % | Extinction |
| | Combustion | | Aerosolized | Coefficient |
| | Temperature | Burn Time | During | (EC) @ λ = 600 nm |
| Composition | (° C.) | (sec) | Combustion) | (m²/g) |
| TA/PE | >425 | | <35 | 0.38 ± 0.39 |
| MATEAB | 344 ± 35.6 | 16 ± 0.8 | 73.6 ± 4.4 | 1.88 ± 0.37 |
| | | | Predicted | |
| | | | Theoretical | |
| | | | 89.75% | |
| Example I | 347 ± 16.9 | 16.8 ± 0.25 | 77.2 ± 4.1 | 1.7 ± 0.42 |
| Example II | 340 ± 23.6 | 26 ± 2.94 | 76.2 ± 0.70 | 2.03 ± 0.38 |
| Phthalimide | | | | |
| Modified MATEAB | | | | |
| Example IV | | 21 ± 1.4 | 78.4 ± 0.49 | 1.86 ± 0.38 |
| Guanidine | | | | |
| Carbonate Modified | | | | |
| MATEAB | | | | |

TABLE VII
Effects of Guandine Carbonate Addition Upon MATEAB Pyrotechnic
Smoke Formulation Combustion Characteristics

| | Max Temp | Burn Time | | EC @ 600 nm |
|---|---|---|---|---|
| Formulation | (° C.) | (s) | % Yield | (M²/g) |
| TA/PE | — | — | — | 0.38 ± 0.39 |
| MATEAB | 344 ± 35.6 | 16 ± 0.8 | 73.6 ± 4.4 | 1.88 ± 0.37 |
| | | | Theoretical | |
| | | | 89.75% | |
| MATEAB + | — | 21 ± 1.4 | 78.4 ± 0.49 | 1.86 ± 0.38 |
| 3 wt. % | | | | |
| Guanidine | | | | |
| carbonate | | | | |
| MATEAB + | 352 ± 19 | 33 ± 8 | 79.2 ± 0.54 | 1.84 ± 0.36 |
| 5.5 wt. % | | | | |
| Guanidine | | | | |
| carbonate | | | | |

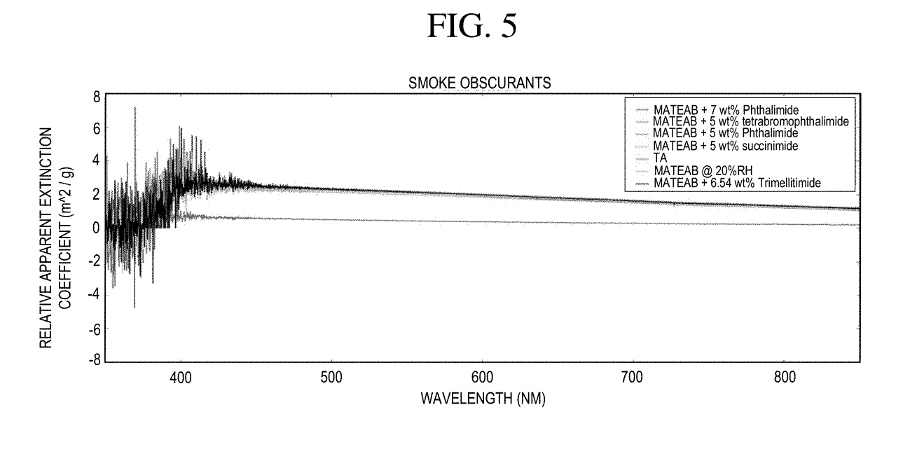

TABLE VIII
Effects of Various Additives upon Peak Combustion Exotherms
of Candidate Smoke Pellet Formulations

| | | Number |
|---|---|---|
| | | of Pellet |
| | Average | Burn |
| Formulation | Peak Temp (° C.) | Trials |
| MATEAB + 5 wt % Sodium Bicarbonate | 413.7 ± 13.6 | 4 |
| MPTEAB (Example I) | 386.8 ± 8.5 | 4 |
| MATTEAB (Table IV) | 356 ± 12.3 | 5 |
| MATEAB 5 wt % Aluminum trihydrate | 355.8 ± 16.7 | 4 |
| MATEAB with 5 wt % phthalimide & | 343.8 ± 6 | 4 |
| 5 wt % PEPA | | |
| MATEAB + 5 wt % phthalimide | 340.3 ± 26 | 4 |
| MATEAB + 3.5 wt % Guanidine carbonate | 338 ± 7.6 | 4 |
| MATEAB + 5 wt % Magnesium carbonate | 331.9 ± 31.4 | 4 |
| MATEAB + 5 wt % Tetrabromophthalimide | 322.3 ± 10.8 | 4 |
| MATEAB + 10 wt % PEPA phosphate | 297.3 ± 10.9 | 4 |
| ester (FIG. 5) | | |

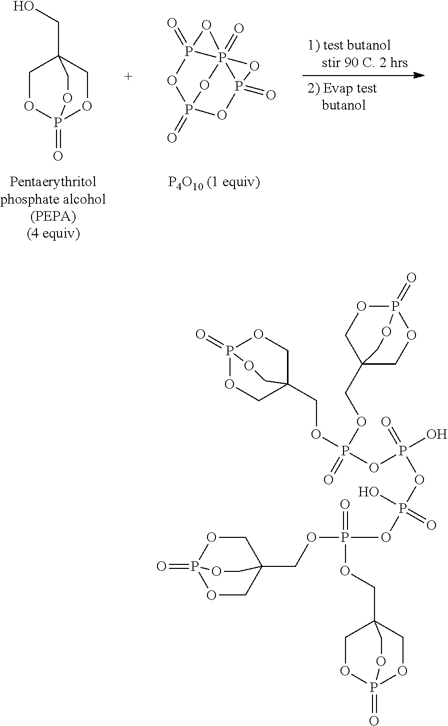

TABLE IX
Candidate MATEAB + PEPA Phosphate Ester Smoke Formulation

| MATEAB + PEPA phosphate ester | |
|---|---|
| Compound | Wt % |
| Potassium Chlorate | 30.70 |
| Sucrose | 12.08 |
| Melamine | 19.89 |
| TEAB | 15.26 |
| Acetoguanamine | 12.99 |
| PEPA phosphate ester (Fig.) | 9.080 |

TABLE X
Comparison Between MATEAB PEPA Phosphate Ester
Modified MATEAB versus TA & MATEAB Smoke Properties

| | Extinction | | | |
|---|---|---|---|---|
| | Coefficient (EC) | Max | | Burn |
| | @ λ = 600 nm | Temp | Percent | Time |
| Formulation | (M²/g) | (° C.) | Yield | (s) |
| TA | 0.38 ± 0.04 | >425 | — | — |
| MATEAB | 1.81 ± 0.07 | 356 ± 12 | 73.6 ± 4.4 | 16 ± 0.8 |
| MATEAB + | 1.84 ± 0.05 | 297 ± 11 | 70 ± 2.5 | 11.8 ± 1 |
| PEPA | | | | |
| phosphate ester | | | | |

TABLE XI
Table XI. Comparison Between Guanidine Phosphate versus TA & MATEAB
Smoke Properties

| | Relative Apparent | | | |
|---|---|---|---|---|
| | Extinction | Peak | Yield Aerosolized | |
| | Coefficient (EC) | Combustion | During | |
| | @ λ = 600 nm | Temperature | Combustion (Mass | Burn Time |
| Formulation | (M²/g) | (° C.) | %) | (s) |
| TA | 0.38 ± 0.04 | >425 | — | — |
| MATEAB | 1.81 ± 0.07 | 356 ± 12 | 73.6 ± 4.4 | 16 ± 0.8 |
| MATEAB + | 1.84 ± 0.05 | 297 ± 11 | 70 ± 2.5 | 11.8 ± 1 |
| 10 wt % PEPA | | | | |
| phosphate ester | | | | |
| MATEAB + | 1.86 ± 0.01 | | | |
| 5 wgt. % | | | | |
| Guanidine | | | | |
| phosphate dibasic | | | | |
| (TCI) | | | | |
| MATEAB + 5 wgt. | 1.82 ± 0.08 | — | — | — |
| % guanidine | | | | |
| phosphate | | | | |
| monobasic | | | | |
| MATEAB + 5.5 wgt. | 1.84 ± 0.03 | — | 79.2 ± 0.54 | 32.7 |
| % Guanidine | | | | |
| carbonate | | | | |

The following Examples are set forth to describe to persons having ordinary skill in the art the utility of combining one or more of Applicant's oleo resins with one or more of Applicant's MATEAB obscurant compositions.

### Example 6

A blend comprising Applicant's Pyrotechnic Composition of Example I with Applicant's Nonivamide Maleic Anhydride Adduct 1 at a blend ratio of 0.5 parts/99.5 to a blend ratio of 99.5 parts/0.5 parts, including all intermediate blend ratios, may be prepared and utilized as an Enhanced Pyrotechnic Composition.

### Example 7

A blend comprising Applicant's Pyrotechnic Composition of Example I with Applicant's Nonivamide Phthalic Anhydride Adduct 2 at a blend ratio of 0.5 parts/99.5 to a blend ratio of 99.5 parts/0.5 parts, including all intermediate blend ratios, may be prepared and utilized as an Enhanced Pyrotechnic Composition.

### Example 8

A blend comprising Applicant's Pyrotechnic Composition of Example I with Applicant's Nonivamide Succinic Anhydride Adduct 3 at a blend ratio of 0.5 parts/99.5 to a blend ratio of 99.5 parts/0.5 parts, including all intermediate blend ratios, may be prepared and utilized as an Enhanced Pyrotechnic Composition.

### Example 9

A blend comprising Applicant's Pyrotechnic Composition of Example II with Applicant's Nonivamide Maleic Anhydride Adduct 1 at a blend ratio of 0.5 parts/99.5 to a blend ratio of 99.5 parts/0.5 parts, including all intermediate blend ratios, may be prepared and utilized as an Enhanced Pyrotechnic Composition.

### Example 10

A blend comprising Applicant's Pyrotechnic Composition of Example II with Applicant's Nonivamide Phthalic Anhydride Adduct 2 at a blend ratio of 0.5 parts/99.5 to a blend ratio of 99.5 parts/0.5 parts, including all intermediate blend ratios, may be prepared and utilized as an Enhanced Pyrotechnic Composition.

### Example 11

A blend comprising Applicant's Pyrotechnic Composition of Example II with Applicant's Nonivamide Succinic Anhydride Adduct 3 at a blend ratio of 0.5 parts/99.5 to a blend ratio of 99.5 parts/0.5 parts, including all intermediate blend ratios, may be prepared and utilized as an Enhanced Pyrotechnic Composition.

### Example 12

A blend comprising Applicant's Pyrotechnic Composition of Example III with Applicant's Nonivamide Maleic Anhydride Adduct 1 at a blend ratio of 0.5 parts/99.5 to a blend ratio of 99.5 parts/0.5 parts, including all intermediate blend ratios, may be prepared and utilized as an Enhanced Pyrotechnic Composition.

### Example 13

A blend comprising Applicant's Pyrotechnic Composition of Example III with Applicant's Nonivamide Phthalic Anhydride Adduct 2 at a blend ratio of 0.5 parts/99.5 to a blend ratio of 99.5 parts/0.5 parts, including all intermediate blend ratios, may be prepared and utilized as an Enhanced Pyrotechnic Composition.

### Example 14

A blend comprising Applicant's Pyrotechnic Composition of Example III with Applicant's Nonivamide Succinic Anhydride Adduct 3 at a blend ratio of 0.5 parts/99.5 to a blend ratio of 99.5 parts/0.5 parts, including all intermediate blend ratios, may be prepared and utilized as an Enhanced Pyrotechnic Composition.

### Example 15

A blend comprising Applicant's Pyrotechnic Composition of Example IV with Applicant's Nonivamide Maleic Anhydride Adduct 1 at a blend ratio of 0.5 parts/99.5 to a blend ratio of 99.5 parts/0.5 parts, including all intermediate blend ratios, may be prepared and utilized as an Enhanced Pyrotechnic Composition.

### Example 16

A blend comprising Applicant's Pyrotechnic Composition of Example IV with Applicant's Nonivamide Phthalic Anhydride Adduct 2 at a blend ratio of 0.5 parts/99.5 to a blend ratio of 99.5 parts/0.5 parts, including all intermediate blend ratios, may be prepared and utilized as an Enhanced Pyrotechnic Composition.

### Example 17

A blend comprising Applicant's Pyrotechnic Composition of Example IV with Applicant's Nonivamide Succinic Anhydride Adduct 3 at a blend ratio of 0.5 parts/99.5 to a blend ratio of 99.5 parts/0.5 parts, including all intermediate blend ratios, may be prepared and utilized as an Enhanced Pyrotechnic Composition.

### Example 18

A blend comprising Applicant's Pyrotechnic Composition of Example V with Applicant's Nonivamide Maleic Anhydride Adduct 1 at a blend ratio of 0.5 parts/99.5 to a blend ratio of 99.5 parts/0.5 parts, including all intermediate blend ratios, may be prepared and utilized as an Enhanced Pyrotechnic Composition.

### Example 19

A blend comprising Applicant's Pyrotechnic Composition of Example V with Applicant's Nonivamide Phthalic Anhydride Adduct 2 at a blend ratio of 0.5 parts/99.5 to a blend ratio of 99.5 parts/0.5 parts, including all intermediate blend ratios, may be prepared and utilized as an Enhanced Pyrotechnic Composition.

### Example 20

A blend comprising Applicant's Pyrotechnic Composition of Example V with Applicant's Nonivamide Succinic Anhydride Adduct 3 at a blend ratio of 0.5 parts/99.5 to a blend ratio of 99.5 parts/0.5 parts, including all intermediate blend ratios, may be prepared and utilized as an Enhanced Pyrotechnic Composition.

While the preferred embodiments of the present disclosure have been described and illustrated in detail, it should be apparent that modifications and adaptations to those embodiments may occur to one skilled in the art without departing from the scope of the present disclosure.

### Example 21

A blend comprising Applicant's Pyrotechnic Composition of Example I with 1,2-bis{(2-oxo-1,3-dioxolan-4-yl)methyl benzene-1,2-dicarboxylate at a blend ratio of 0.5 parts/99.5 to a blend ratio of 99.5 parts/0.5 parts, including all intermediate blend ratios, may be prepared and utilized as an Enhanced Pyrotechnic Composition.

### Example 22

A blend comprising Applicant's Pyrotechnic Composition of Example I with (2Z)-4-oxo-4-[(2-oxo-1,3-dioxolan-4-yl)methyloxy]but-2-enoic acid-2-benzoic acid at a blend ratio of 0.5 parts/99.5 to a blend ratio of 99.5 parts/0.5 parts, including all intermediate blend ratios, may be prepared and utilized as an Enhanced Pyrotechnic Composition.

### Example 23

A blend comprising Applicant's Pyrotechnic Composition of Example I with (2Z)-4-oxo-4-[(2-oxo-1,3-dioxolan-4-yl)methyloxy]but-2-enoic acid at a blend ratio of 0.5 parts/99.5 to a blend ratio of 99.5 parts/0.5 parts, including all intermediate blend ratios, may be prepared and utilized as an Enhanced Pyrotechnic Composition.

### Example 24

A blend comprising Applicant's Pyrotechnic Composition of Example I with glycerine carbonate ester copolymer at a blend ratio of 0.5 parts/99.5 to a blend ratio of 99.5 parts/0.5 parts, including all intermediate blend ratios, may be prepared and utilized as an Enhanced Pyrotechnic Composition

### Example 25

A blend comprising Applicant's Pyrotechnic Composition of Example II with 1,2-bis {(2-oxo-1,3-dioxolan-4-yl)methyl benzene-1,2-dicarboxylate at a blend ratio of 0.5 parts/99.5 to a blend ratio of 99.5 parts/0.5 parts, including all intermediate blend ratios, may be prepared and utilized as an Enhanced Pyrotechnic Composition.

### Example 26

A blend comprising Applicant's Pyrotechnic Composition of Example II with (2Z)-4-oxo-4-[(2-oxo-1,3-dioxolan-4-yl)methyloxy]but-2-enoic acid-2-benzoic acid at a blend ratio of 0.5 parts/99.5 to a blend ratio of 99.5 parts/0.5 parts, including all intermediate blend ratios, may be prepared and utilized as an Enhanced Pyrotechnic Composition.

### Example 27

A blend comprising Applicant's Pyrotechnic Composition of Example II with (2Z)-4-oxo-4-[(2-oxo-1,3-dioxolan-4-yl)methyloxy]but-2-enoic acid at a blend ratio of 0.5 parts/99.5 to a blend ratio of 99.5 parts/0.5 parts, including all intermediate blend ratios, may be prepared and utilized as an Enhanced Pyrotechnic Composition.

### Example 28

A blend comprising Applicant's Pyrotechnic Composition of Example II with glycerine carbonate ester copolymer at a blend ratio of 0.5 parts/99.5 to a blend ratio of 99.5 parts/0.5 parts, including all intermediate blend ratios, may be prepared and utilized as an Enhanced Pyrotechnic Composition

### Example 29

A blend comprising Applicant's Pyrotechnic Composition of Example III with 1,2-bis{(2-oxo-1,3-dioxolan-4-yl)methyl benzene-1,2-dicarboxylate at a blend ratio of 0.5 parts/99.5 to a blend ratio of 99.5 parts/0.5 parts, including all intermediate blend ratios, may be prepared and utilized as an Enhanced Pyrotechnic Composition.

### Example 30

A blend comprising Applicant's Pyrotechnic Composition of Example III with (2Z)-4-oxo-4-[(2-oxo-1,3-dioxolan-4-yl)methyloxy]but-2-enoic acid-2-benzoic acid at a blend ratio of 0.5 parts/99.5 to a blend ratio of 99.5 parts/0.5 parts, including all intermediate blend ratios, may be prepared and utilized as an Enhanced Pyrotechnic Composition.

### Example 31

A blend comprising Applicant's Pyrotechnic Composition of Example III with (2Z)-4-oxo-4-[(2-oxo-1,3-dioxolan-4-yl)methyloxy]but-2-enoic acid at a blend ratio of 0.5 parts/99.5 to a blend ratio of 99.5 parts/0.5 parts, including all intermediate blend ratios, may be prepared and utilized as an Enhanced Pyrotechnic Composition.

### Example 32

A blend comprising Applicant's Pyrotechnic Composition of Example III with glycerine carbonate ester copolymer at a blend ratio of 0.5 parts/99.5 to a blend ratio of 99.5 parts/0.5 parts, including all intermediate blend ratios, may be prepared and utilized as an Enhanced Pyrotechnic Composition

### Example 33

A blend comprising Applicant's Pyrotechnic Composition of Example IV with 1,2-bis{(2-oxo-1,3-dioxolan-4-yl)methyl benzene-1,2-dicarboxylate at a blend ratio of 0.5 parts/99.5 to a blend ratio of 99.5 parts/0.5 parts, including all intermediate blend ratios, may be prepared and utilized as an Enhanced Pyrotechnic Composition.

### Example 34

A blend comprising Applicant's Pyrotechnic Composition of Example IV with (2Z)-4-oxo-4-[(2-oxo-1,3-dioxolan-4-yl)methyloxy]but-2-enoic acid-2-benzoic acid at a blend ratio of 0.5 parts/99.5 to a blend ratio of 99.5 parts/0.5 parts, including all intermediate blend ratios, may be prepared and utilized as an Enhanced Pyrotechnic Composition.

### Example 35

A blend comprising Applicant's Pyrotechnic Composition of Example IV with (2Z)-4-oxo-4-[(2-oxo-1,3-dioxolan-4-yl)methyloxy]but-2-enoic acid at a blend ratio of 0.5 parts/99.5 to a blend ratio of 99.5 parts/0.5 parts, including all intermediate blend ratios, may be prepared and utilized as an Enhanced Pyrotechnic Composition.

### Example 36

A blend comprising Applicant's Pyrotechnic Composition of Example IV with glycerine carbonate ester copolymer at a blend ratio of 0.5 parts/99.5 to a blend ratio of 99.5 parts/0.5 parts, including all intermediate blend ratios, may be prepared and utilized as an Enhanced Pyrotechnic Composition.

### Example 37

A blend comprising Applicant's Pyrotechnic Composition of Example V with 1,2-bis{(2-oxo-1,3-dioxolan-4-yl)methyl benzene-1,2-dicarboxylate at a blend ratio of 0.5 parts/99.5 to a blend ratio of 99.5 parts/0.5 parts, including all intermediate blend ratios, may be prepared and utilized as an Enhanced Pyrotechnic Composition.

### Example 38

A blend comprising Applicant's Pyrotechnic Composition of Example V with (2Z)-4-oxo-4-[(2-oxo-1,3-dioxolan-4-yl)methyloxy]but-2-enoic acid-2-benzoic acid at a blend ratio of 0.5 parts/99.5 to a blend ratio of 99.5 parts/0.5 parts, including all intermediate blend ratios, may be prepared and utilized as an Enhanced Pyrotechnic Composition.

### Example 39

A blend comprising Applicant's Pyrotechnic Composition of Example V with (2Z)-4-oxo-4-[(2-oxo-1,3-dioxolan-4-yl)methyloxy]but-2-enoic acid at a blend ratio of 0.5 parts/99.5 to a blend ratio of 99.5 parts/0.5 parts, including all intermediate blend ratios, may be prepared and utilized as an Enhanced Pyrotechnic Composition.

### Example 40

A blend comprising Applicant's Pyrotechnic Composition of Example V with glycerine carbonate ester copolymer at a blend ratio of 0.5 parts/99.5 to a blend ratio of 99.5 parts/0.5 parts, including all intermediate blend ratios, may be prepared and utilized as an Enhanced Pyrotechnic Composition

While the preferred embodiments of the present invention have been illustrated in detail, it should be apparent that modifications and adaptations to those embodiments may occur to one skilled in the art without departing from the scope of the present invention.

I claim:

1. An enhanced pyrotechnic composition, comprising:
an obscurant;
a fuel;
an oxidizer; and
a nonivamide-maleic anhydride adduct having a structure:

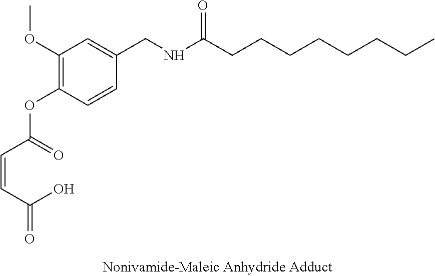 [SMILES: CCCCCCCCC(=O)NCc1ccc(OC(=O)/C=C\C(=O)O)c(OC)c1]

2. The enhanced pyrotechnic composition of claim 1, wherein said obscurant comprises phthalimide.

3. The enhanced pyrotechnic composition of claim 1, wherein said obscurant comprises melamine.

4. The enhanced pyrotechnic composition of claim 1, wherein said obscurant comprises acetoguanamine.

5. The enhanced pyrotechnic composition of claim 1, wherein said obscurant comprises guanidine carbonate.

6. The enhanced pyrotechnic composition of claim 1, wherein said obscurant comprises trimellitimide.

7. The enhanced pyrotechnic composition of claim 1, wherein said obscurant comprises acetogunamine.

8. An enhanced pyrotechnic composition, comprising:
an obscurant;
a fuel;
an oxidizer; and
a nonivamide-phthalic anhydride adduct having a structure:

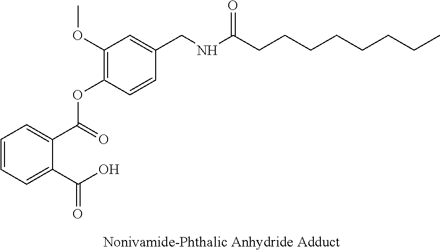 [SMILES: CCCCCCCCC(=O)NCc1ccc(OC(=O)c2ccccc2C(=O)O)c(OC)c1]

9. The enhanced pyrotechnic composition of claim 8, wherein said obscurant comprises phthalimide.

10. The enhanced pyrotechnic composition of claim 8, wherein said obscurant comprises melamine.

11. The enhanced pyrotechnic composition of claim 8, wherein said obscurant comprises acetoguanamine.

12. The enhanced pyrotechnic composition of claim 8, wherein said obscurant comprises guanidine carbonate.

13. The enhanced pyrotechnic composition of claim 8, wherein said obscurant comprises trimellitimide.

14. The enhanced pyrotechnic composition of claim 8, wherein said obscurant comprises acetogunamine.

15. An enhanced pyrotechnic composition, comprising:
an obscurant;
a fuel;
an oxidizer; and
a nonivamide-succinic anhydride adduct having a structure:

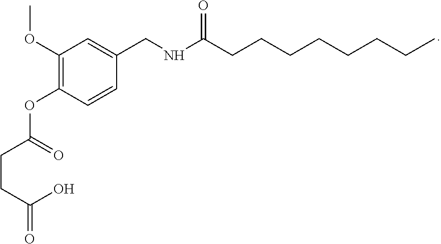 [SMILES: CCCCCCCCC(=O)NCc1ccc(OC(=O)CCC(=O)O)c(OC)c1]

16. The enhanced pyrotechnic composition of claim 8, wherein said obscurant comprises phthalimide.

17. The enhanced pyrotechnic composition of claim 8, wherein said obscurant comprises melamine.

18. The enhanced pyrotechnic composition of claim 8, wherein said obscurant comprises acetoguanamine.

19. The enhanced pyrotechnic composition of claim 8, wherein said obscurant comprises guanidine carbonate.

20. The enhanced pyrotechnic composition of claim 8, wherein said obscurant comprises trimellitimide.

21. The enhanced pyrotechnic composition of claim 8, wherein said obscurant comprises acetogunamine.

22. An enhanced pyrotechnic composition, comprising:
an obscurant;
a fuel;
an oxidizer; and
1,2-bis{(2-oxo-1,3-dioxolan-4-yl)methyl benzene-1,2-dicarboxylate.

23. The enhanced pyrotechnic composition of claim 22, wherein said obscurant comprises phthalimide.

24. The enhanced pyrotechnic composition of claim 22, wherein said obscurant comprises melamine.

25. The enhanced pyrotechnic composition of claim 22, wherein said obscurant comprises acetoguanamine.

26. The enhanced pyrotechnic composition of claim 22, wherein said obscurant comprises guanidine carbonate.

27. The enhanced pyrotechnic composition of claim 22, wherein said obscurant comprises trimellitimide.

28. The enhanced pyrotechnic composition of claim 22, wherein said obscurant comprises acetogunamine.

29. An enhanced pyrotechnic composition, comprising:
an obscurant;
a fuel;
an oxidizer; and
(2Z)-4-oxo-4-[(2-oxo-1,3-dioxolan-4-yl)methyloxy]but-2-enoic acid-2-benzoic acid.

30. The enhanced pyrotechnic composition of claim 29, wherein said obscurant comprises phthalimide.

31. The enhanced pyrotechnic composition of claim 29, wherein said obscurant comprises melamine.

32. The enhanced pyrotechnic composition of claim 29, wherein said obscurant comprises acetoguanamine.

33. The enhanced pyrotechnic composition of claim 29, wherein said obscurant comprises guanidine carbonate.

34. The enhanced pyrotechnic composition of claim 29, wherein said obscurant comprises trimellitimide.

35. The enhanced pyrotechnic composition of claim 29, wherein said obscurant comprises acetogunamine.

36. An enhanced pyrotechnic composition, comprising:
an obscurant;
a fuel;
an oxidizer; and
glycerine carbonate ester copolymer.

37. The enhanced pyrotechnic composition of claim 36, wherein said obscurant comprises phthalimide.

38. The enhanced pyrotechnic composition of claim 36, wherein said obscurant comprises melamine.

39. The enhanced pyrotechnic composition of claim 36, wherein said obscurant comprises acetoguanamine.

40. The enhanced pyrotechnic composition of claim 36, wherein said obscurant comprises guanidine carbonate.

41. The enhanced pyrotechnic composition of claim 36, wherein said obscurant comprises trimellitimide.

42. The enhanced pyrotechnic composition of claim 36, wherein said obscurant comprises acetogunamine.
